(19) United States
(12) Patent Application Publication
(10) Pub. No.: US 2021/0077371 A1
(43) Pub. Date: Mar. 18, 2021

(54) OILY SOLID CLEANSING COSMETIC

(71) Applicant: TOKIWA CORPORATION, Gifu (JP)

(72) Inventor: Kenji KOKAI, Saitama (JP)

(73) Assignee: TOKIWA CORPORATION, Gifu (JP)

(21) Appl. No.: 17/014,411

(22) Filed: Sep. 8, 2020

(30) Foreign Application Priority Data
Sep. 12, 2019 (JP) 2019-166255
Oct. 18, 2019 (JP) 2019-191388

(51) Int. Cl. A61K 8/37; A61K 8/73; A61K 8/02; A61K 8/81; A61Q 1/14; A61K 8/92

(52) U.S. Cl. CPC A61K 8/37; A61K 8/738; A61K 8/922; A61K 8/8111; A61Q 1/14; A61K 8/0216

(57) ABSTRACT

The oily solid cleansing cosmetic contains: (A) hydrogenated castor oil, (B) a hydrocarbon-based wax, (C) an ester oil whose viscosity at 25° C. is 50 mPa·s or less, and (D) a nonionic surfactant, wherein the content of the component (A) is 1% to 5% by mass, the content of the component (B) is 1% to 5% by mass, the content of the component (C) is 60% by mass or more, and the total content of the component (A) and the component (B) is 4% to 7.5% by mass.

### TECHNICAL FIELD

The present invention relates to an oily solid cleansing cosmetic.

### BACKGROUND

In recent years, numerous makeup cosmetics having excellent makeup durability have been developed. Therefore, for the removal of makeup, oily cleansing cosmetics having favorable compatibility with makeup cosmetics (makeup compatibility) are suitable. Oily cleansing cosmetics are generally provided in a liquid or solid form.

A liquid oily cleansing cosmetic contains liquid oil as a main component, and it is known that the compatibility with makeup cosmetics is especially excellent and the cleansing effect is high. However, since a liquid oily cleansing cosmetic has low viscosity there is a problem that the cosmetic easily drips at the time of use, and there is also a problem that the friction movement during use is likely to be directly transferred to the skin, and the burden or stimulation to the skin is strongly felt.

On the other hand, in a solid oily cleansing cosmetic, solid oil such as wax is incorporated therein in addition to liquid oil, and as liquid oil is incorporated into a card house structure formed by solid oil, an oil gel structure in which the fluidity of the liquid oil is suppressed is formed. Such an oily solid cleansing cosmetic can be easily handled as compared to liquid cleansing cosmetics, and since an oily solid cleansing cosmetic gives shearing stress to the oil gel structure during use, parts of the structure are disintegrated, and the liquid oil can be caused to spread out on the skin and become compatible with makeup cosmetics. Enhancement of the spread-out and the makeup compatibility of the oily solid cleansing cosmetics has also been investigated, and for example, in Japanese Unexamined Patent Publication No. 2017-095375, an oily solid cleansing cosmetic in which a polysaccharide fatty acid ester and a solid anionic surfactant are further incorporated in addition to a liquid oil and a solid oil, has been suggested.

### SUMMARY

In conventional oily solid cleansing cosmetics, since an oil gel structure becomes rigid at the time of low temperature such as the winter season, the cleansing cosmetics cannot be easily taken out from the containers, and the compatibility with makeup cosmetics may be deteriorated. When the content of the solid oil is reduced and the oil gel structure is made weak in order to improve the usability at low temperature, storage stability becomes insufficient because the hardness is decreased in a temperature cycle test, or the like. Therefore, it is difficult to achieve both usability and storage stability in a well-balanced manner, simply by changing the amount of the solid oil only and adjusting the strength of the oil gel structure. Incidentally, there is also a technique of reinforcing the oil gel structure by using an oil gelling agent in combination with the cosmetic of the above-described patent literature; however, the technique cannot be said to be necessarily satisfying in view of usability because the makeup compatibility is deteriorated, sliminess is felt, and the like.

The present invention was achieved in view of the circumstances described above, and it is an object of the invention to provide an oily solid cleansing cosmetic that can be easily taken out from, the container, has excellent compatibility with makeup cosmetics, and can achieve both satisfactory usability with less sliminess and sufficient storage stability that makes the hardness not easily changeable.

In order to solve the problems described above, the present invention provides an oily solid cleansing cosmetic containing: (A) hydrogenated castor oil; (B) a hydrocarbon-based wax; (C) an ester oil whose viscosity at 25° C. is 50 mPa·s or less; and (D) a nonionic surfactant, wherein the content of the component (A) is 1% to 5% by mass, the content of the component (B) is 1% to 5% by mass, the content of the component (C) is 60% by mass or more, and the total content of the component (A) and the component (B) is 4% to 7.5% by mass.

As the oily solid cleansing cosmetic of the present invention has the above-described configuration, the oily solid cleansing cosmetic can be easily taken out from the container, has excellent compatibility with makeup cosmetics, and can achieve both satisfactory usability with less sliminess and sufficient storage stability that makes the hardness not easily changeable.

The inventors of the present invention consider the reason why the above-described effects are obtained, as follows. By incorporating hydrogenated castor oil, the oil gel structure formed by including a hydrocarbon-based wax and the above-described specific ester oil is stabilized. Furthermore, it is considered that as the various components are included at the above-described particular contents, storage stability is sufficient, the oil gel structure is disintegrated with light shearing stress at the time of use, and a sherbet-like structure in which the ester oil easily spreads out without feeling sliminess, can be formed. Furthermore, the present inventors think that the specific ester oil having excellent makeup compatibility and spreading is also a factor by which satisfactory usability is obtainable.

With regard to the oily solid cleansing cosmetic of the present invention, from the viewpoints of makeup compatibility and spread-out, it is preferable that the total content of the component (A), the component (B), the component (C), and the component (D) is 97% by mass or more.

From the viewpoint of being easily compatible with makeup cosmetics and obtaining usability with less sliminess, it is preferable that 50% by mass or more of the component (C) is an ester oil whose viscosity at 25° C. is 20 mPa·s or less.

From the viewpoint of shape retainability, it is preferable that the component (B) is a polyethylene wax at a melting point of 70° C. to 110° C.

From the viewpoint of obtaining more satisfactory usability while sufficiently maintaining storage stability, it is preferable that the mass ratio (A)/(B) between the component (A) and the component (B) is 1/5 to 1/1.

With regard to the oily solid cleansing cosmetic of the present invention, from the viewpoint of being easily taken out from the container and obtaining usability with further less sliminess, it is preferable that the hardness at 25° C. is 0.3 to 1.2 N.

According to the present invention, there can be provided an oily solid cleansing cosmetic that can be easily taken out from the container, has excellent compatibility with makeup cosmetics, and can achieve both satisfactory usability with less sliminess and sufficient storage stability that makes the hardness not easily changeable.

### DETAILED DESCRIPTION

The oily solid cleansing cosmetic of the present embodiment contains: (A) hydrogenated castor oil (hereinafter, may be referred to as component (A)); (B) a hydrocarbon-based wax (hereinafter, may be referred to as component (B)); (C) an ester oil whose viscosity at 25° C. is 50 mPa·s or less (hereinafter, may be referred to as component (C)); and (D) a nonionic surfactant (hereinafter, may be referred to as component (D)).

<(A) Hydrogenated Castor Oil>

Regarding the hydrogenated castor oil (hardened castor oil), any hydrogenated castor oil can be used without particular limitation as long as it is used for perfumery and cosmetics. Regarding the hydrogenated castor oil, commercially available products such as Hardened Castor Oil A (manufactured by ITOH OIL CHEMICALS CO., LTD., product name), Castor Wax A Flakes (manufactured by NOF Corp., product name), and HIMAKO (manufactured by KF TRADING CO., LTD., product name) can be used.

Regarding the component (A), one kind thereof can be used alone, or two or more kinds thereof can be used in combination.

The content of the component (A) in the oily solid cleansing cosmetic can be set to 1% to 5% by mass based on the total amount of the cosmetic, and may be 1% to 4% by mass, may be 1% to 3% by mass, or may be 1% to 2% by mass.

<(B) Hydrocarbon-Based Wax>

Examples of the hydrocarbon-based wax include polyethylene wax, paraffin wax, microcrystalline wax, Fischer-Tropsch wax, Ceresin, ozokerite, and the like.

Regarding the component (B), one kind thereof can be used alone, or two or more kinds thereof can be used in combination.

From the viewpoints of shape retainability and storage stability, the hydrocarbon-based wax is such that the melting point is preferably 70° C. to 110° C., more preferably 80° C. to 110° C., and even more preferably 80° C. to 105° C.

The melting point according to the present specification means a value measured according to the following method.

First, about 5 mg of a sample is weighed and placed in an aluminum sample pan. An aluminum cover is attached to this pan, and the pan is installed in a differential scanning calorimeter “DSC7020” (manufactured by Hitachi High-Tech Science Corp., product name). Using an electric cooling unit “POLYSCIENCE” (manufactured by Hitachi High-Tech Science Corp., product name), a sample and a reference sample are maintained for one minute at 0° C. under a nitrogen gas flow rate of 30 to 50 mL/min. Subsequently, a melt endothermic curve is obtained by raising the temperature from 0° C. to 120° C. at a rate of temperature increase of 10° C./min, lowering the temperature from 120° C. to 0° C. under a condition for temperature decrease of −10° C./min, and raising the temperature again from 0° C. to 120° C. at a rate of temperature increase of 10° C./min. The peak temperature of the melt endothermic curve in the second temperature increase at this time is designated as the melting point.

From the viewpoint of storage stability, it is preferable that the oily solid cleansing cosmetic includes a polyethylene wax having a melting point of 70° C. to 110° C., and preferably 80° C. to 105° C.

The content of the component (B) in the oily solid cleansing cosmetic can be set to 1% to 5% by mass based on the total amount of the cosmetic, and the content may be 2% to 5% by mass, may be 3% to 5% by mass, or may be 4% to 6% by mass.

From the viewpoint of achieving both usability and storage stability at a high level, the total content of the component (A) and the component (B) is preferably 4% to 7.5% by mass, more preferably 4% to 7% by mass, and even more preferably 4% to 6% by mass, based on the total amount of the cosmetic.

Furthermore, from the viewpoint of obtaining more satisfactory usability while sufficiently maintaining storage stability, the mass ratio (A)/(B) between the component (A) and the component (B) is preferably 1/5 to 1/1, more preferably 1/5 to 1/3, and even more preferably 1/5 to 1/2.

<(C) Ester Oil Whose Viscosity at 25° C. is 50 mPa·s or Less>

Examples of the ester oil whose viscosity at 25° C. is 50 mPa·s or less include triethylhexanoin, isopropyl palmitate, ethylhexyl palmitate, h exyldecyl ethylhexanoate, cetyl ethylhexanoate, isononyl isononanoate, isotridecyl isononanoate, isopropyl isostearate, isostearyl isostearate, neopentyl glycol diethylhexanoate, glyceryl tri(caprylate/caprate), neopentyl glycol dicaprate, propanediol diisostearate, trimethylolpropane triethylhexanoate, isopropyl myristate, octyl dodecyl myristate, and the like.

The viscosity at 25° C. of the ester oil according to the present specification refers to a value at the time of measuring the viscosity for a sample at 25° C. using a type B viscometer under the conditions of a BL adaptor and a speed of rotation of 12 rpm.

Regarding the component (C), one kind thereof can be used alone, or two or more kinds thereof can be used in combination.

From the viewpoint of makeup compatibility, the content of the component (C) in the oily solid cleansing cosmetic is preferably 60% by mass or more, more preferably 70% by mass or more, even more preferably 75% by mass or more, and still more preferably 80% by mass or more, based on the total amount of the cosmetic. From the viewpoints of shape retainability and storage stability, the content is preferably 95% by mass or less, more preferably 90% by mass or less, and even more preferably 85% by mass or less.

From the viewpoint that it is easily compatible with makeup cosmetics and usability with further less sliminess is easily obtainable, it is preferable that 50% by mass or more, more preferably 60% by mass or more, and even more preferably 70% by mass or more, of the component (C) is an ester oil whose viscosity at 25° C. is 20 mPa·s or less, and more preferably an ester oil whose viscosity at 25° C. is 15 mPa·s or less.

Examples of the ester oil whose viscosity at 25° C. is 20 mPa·s or less include ethylhexyl palmitate, isopropyl myristate, isopropyl isostearate, cetyl ethylhexanoate, and the like.

From the viewpoints of makeup compatibility and storage stability, it is preferable that the oily solid cleansing cosmetic contains, as the component (C), a first ester oil whose viscosity at 25° C. is 1 to 15 mPa·s, more preferably 5 to 15 mPa·s, and even more preferably 7 to 13 mPa·s; and a second ester oil whose viscosity at 25° C. is 16 to 50 mPa·s, more preferably 20 to 50 mPa·s, even more preferably 20 to 40 mPa-s, still more preferably 25 to 35 mPa·s, and particularly preferably 25 to 32 mPa·s. From the viewpoint of satisfying the makeup compatibility, storage stability; and the absence of sliminess at a higher level, regarding the second ester oil, the viscosity at 25° C. is preferably 25 to 35 mPa·s, and more preferably 25 to 32 mPa·s.

The mass ratio between the first ester oil and the second ester oil, (first ester oil)/(second ester oil), is preferably 1/1 to 4/1, more preferably 2/1 to 4/1, and even more preferably 3/1 to 4/1, from the viewpoint of reducing sliminess.

<(D) Nonionic Surfactant>

Examples of the nonionic surfactant include a polyoxyalkylene alkyl ether, a glycerin alkyl ether, a glycerin fatty acid ester, a polyglycerin fatty acid ester, a sorbitan fatty acid ester, alkylene glycol adducts of those, a polyalkylene glycol fatty acid ester, a polyglycerin-modified silicone, a polyether-modified silicone, and the like.

Regarding the component (D), one kind thereof can be used alone, or two or more kinds thereof can be used in combination.

Regarding the component (D), from the viewpoint of the ease of washing away with water or lukewarm water after cleansing, the HLB is preferably 7 or higher, and from the viewpoint of reducing the taut feeling after washing away, the HLB is preferably 14 or lower.

With regard to the oily solid cleansing cosmetic of the present embodiment, from the viewpoints of makeup compatibility spreading, the total content of the component (A), the component (B), the component (C), and the component (D) may be 97% by mass or more, may be 97.5% by mass or more, may be 98% by mass or more, may be 98.5% by mass or more, or may be 99% by mass or more, based on the total amount of the cosmetic.

In the oily solid cleansing cosmetic of the present embodiment, in addition to the various components described above, other components that are usually used in cosmetic products, for example, a liquid oil other than the component (C), an anionic surfactant, a cationic surfactant, an amphoteric surfactant, a humectant, a viscosity adjusting agent, an antiseptic agent, a pH adjusting agent, a chelating agent, an ultraviolet absorber, vitamins, beauty ingredients, an oxidation inhibitor, fragrance, and the like can be appropriately incorporated as necessary, to the extent that does not impair the effects of the present invention.

As the liquid oil other than the component (C), an aliphatic alcohol, a hydrocarbon oil, a silicone oil, an ester oil whose viscosity at 25° C. is more than 50 mPa·s, or the like can be used. Examples of the aliphatic alcohol include aliphatic alcohols that are liquid at 25° C., such as octyldodecanol, hexyldodecanol, isostearyl alcohol, and oleyl alcohol. Examples of the silicone oil include methyl polysiloxane, methyl phenyl polysiloxane, octamethylcyclotetrasiloxane, decamethylcyclopentasiloxane, dodecamethylcyclohexasiloxane, stearoxymethyl polysiloxane, diphenylsiloxy phenyl trimethicone, a fluorine-modified silicone, and the like. Examples of the hydrocarbon oil include liquid paraffin, squalene, hydrogenated polyisobutene, and the like. Examples of the ester oil whose viscosity at 25° C. is more than 50 mPa·s include ethylhexyl hydroxystearate, diisopropyl dilinoleate, trimethylolpropane triisostearate, polyglyceryl-2 triisostearate, polyglyceryl-2 diisostearate, polyglyceryl-2 isostearate, diisostearyl malate, polyglyceryl-3 diisostearate, and the like.

The content of the liquid oil other than the component (C) in the oily solid cleansing cosmetic may be 0.1% to 30% by mass based on the total amount of the cosmetic, from the viewpoint of reducing the taut feeling after washing away.

In a case in which the oily solid cleansing cosmetic includes a liquid oil other than the component (C), from the viewpoint of makeup compatibility, the total content of the component (C) and the liquid oil other than the component (C) is preferably 70% to 95% by mass, and more preferably 80% to 85% by mass, based on the total amount of the cosmetic. Furthermore, from the viewpoint of storage stability, the mass ratio between the component (C) and the liquid oil other than the component (C), (component (C))/(liquid oil other than component (C)), is preferably 2/1 to 20/1, more preferably 2/1 to 10/1, even more preferably 2/1 to 8/1, and still more preferably 2.5/1 to 5/1.

It is preferable that the oily solid cleansing cosmetic of the present embodiment includes one kind or two or more kinds selected from, the group consisting of an aliphatic alcohol and a silicone oil, as the liquid oil other than the component (C).

Regarding the oily solid cleansing cosmetic of the present embodiment, from the viewpoint of being easily taken out from the container and obtaining usability with further less sliminess, the hardness measured using a rheometer (manufactured by RHEOTECH) with the tip shape of the pressure-sensitive shaft: disc having a diameter of 5 mm, the rate of penetration: 6 cm/min, the depth of penetration: 10 mm, and temperature: 25° C. is preferably 0.3 to 1.2 N, more preferably 0.4 to 1.1 N, and even more preferably 0.5 to 1.0 N.

Next, a method for producing an oily solid cleansing cosmetic of the present embodiment will be described.

The method for producing an oily solid cleansing cosmetic of the present embodiment comprises a step of preparing a cosmetic base material containing the above-mentioned component (A), component (B), component (C), and component (D) as well as other components as necessary; and a step of charging the cosmetic base material into a predetermined container and solidifying the cosmetic base material.

Regarding the cosmetic base material, the composition can be appropriately set so as to obtain the oily solid cleansing cosmetic of the present embodiment as described above. The mixing amounts of the various components in the cosmetic base material can also be adjusted similarly to the preferred ranges for the above-mentioned oily solid cleansing cosmetic.

Regarding the preparation of the cosmetic base material, a method of mixing the above-mentioned component (A), component (B), component (C), component (D), and optionally other components while heating the components may be used.

The mixing means is not particularly limited; however, for example, a Disper, a Homomixer, and the like can be used. The heating temperature can be set to a temperature higher than or equal to the melting points of the component (A) and the component (B).

Solidification of the cosmetic base material can be carried out by, for example, a method of cooling the cosmetic base material before charging to room temperature, or the like.

Examples of the predetermined container include a bottle, a tube, a jar, a pouch container, and the like.

### EXAMPLES

Hereinafter, the present invention will be more specifically described by way of Examples; however, the present invention is not intended to be limited to the following Examples.

### Production of Oily Solid Cleansing Cosmetic

### Examples 1 to 15 and Comparative Examples 1 to 7

The various components shown in Tables 1 to 3 were mixed at 105° C. at the proportions (% by mass) indicated in the same tables using a Disper, and the respective oily solid cleansing cosmetics were obtained.

Incidentally, the details of the various components shown in Tables 1 to 3 are as follows.

Hydrogenated castor oil: Melting point 85° C. to 87° C.

Polyethylene wax A: PERFORMALENE 500 POLYETHYLENE (manufactured by Baker Petrolite LLC, product name, melting point 83.8° C.)

Polyethylene wax B: PERFORMALENE 655 POLYETHYLENE (manufactured by Baker Petrolite LLC, product name, melting point 96.7° C.)

Candelilla wax: Melting point 75° C. to 85° C. (catalogue value)

Carnauba wax: Melting point 80° C. to 86° C. (catalogue value)

Ethylhexyl palmitate: Viscosity 11 mPa·s @ 25° C.

Triethylhexanoin: Viscosity 31 mPa·s @ 25° C.

Glyceryl tri(caprylate/caprate): Viscosity 26 mPa·s @ 25° C.

Isostearyl isostearate: Viscosity 36 mPa·s @ 25° C.

Dimethicone: Viscosity 6 mPa·s @ 25° C.

Diphenylsiloxyphenyltrimethicone: Viscosity 15 mPa·s @ 25° C.

(Melting Point of Wax)

The melting point of a wax was measured by the following procedure.

About 5 mg of a sample was weighed and placed in an aluminum sample pan. An aluminum cover was attached to this pan, and the pan was installed in a differential scanning calorimeter “DSC7020” (manufactured by Hitachi High-Tech Science Corp., product name). Using an electric cooling unit “POLYSCIENCE” (manufactured by Hitachi High-Tech Science Corp., product name), a sample and a reference sample were maintained for one minute at 0° C. under a nitrogen gas flow rate of 30 to 50 mL/min. Subsequently, a melt endothermic curve was obtained by raising the temperature from 0° C. to 120° C. at a rate of temperature increase of 10° C./min, lowering the temperature from 120° C. to 0° C. under a condition for temperature decrease of −14° C./min, and raising the temperature again from 0° C. to 120° C. at a rate of temperature increase of 14° C./min. The peak temperature of the melt endothermic curve in the second temperature increase at this time was designated as the melting point.

(Viscosity of Ester Oil)

For a sample at 25° C., the viscosity was measured using a type B viscometer under the conditions of a BL adaptor and a speed of rotation of 12 rpm. Incidentally, the measurement time was set to 1 minute. Furthermore, even for a liquid oil other than an ester oil, the viscosity was measured under similar conditions.

<Evaluation of Oily Solid Cleansing Cosmetic>

For an oily solid cleansing cosmetic, the initial hardness, usability (ease of taking out, makeup compatibility, absence of sliminess), and storage stability were evaluated according to the following evaluation methods. The results are presented in Tables 1 and 2.

(Initial Hardness)

A sample for measurement obtained by charging a cosmetic into an ointment jar having a capacity of 20 mL and leaving the cosmetic to stand overnight at 25° C. was prepared. For this sample for measurement, the hardness was measured using a rheometer (manufactured by RHEOTECH) under the conditions of the tip shape of the pressure-sensitive shaft: disc having a diameter of 5 mm, speed of penetration: 6 cm/min, depth of penetration: 10 mm, and temperature: 25° C.

(Usability: Ease of Taking Out, Make Compatibility, and Absence of Sliminess)

Ten expert panels for cosmetic product evaluation were asked to use samples, and each panel performed a 5-grade evaluation for the ease of taking out from the container, makeup compatibility at the time of being applied on the skin, and the absence of sliminess, according to the following evaluation criteria. Each panel assigned a score for each sample, and the average point of the scores given by all the panels was judged according to the following criteria.

[Evaluation Criteria]

5 points: Very satisfactory

4 points: Satisfactory

3 points: Normal

2 points: Slightly poor

1 point: Poor [Judgment Criteria (Average Point of Scores)]

A: 4.5 or higher

B: 3.5 or higher and lower than 4.5

C: 2.5 or higher and lower than 3.5

D: Lower than 2.5

(Storage Stability)

100 g of a cosmetic was charged into a jar container and was stored for one week in a cyclic constant-temperature tank at 5° C. to 40° C. For the cosmetic after storage, the hardness was measured by a method similar to that for the initial hardness, the change ratio of the hardness to the initial hardness was determined, and the change ratio was judged in 3 grades according to the following criteria.

[Judgment Criteria]

A: The change ratio with respect to the initial hardness is ±0% or higher and 20% or lower.

B: The change ratio with respect to the initial hardness is higher than ±20% and 50% or lower.

C: The change ratio with respect to the initial hardness is higher than ±50%.

TABLE 1

| | | Example | Example | Example | Example | Example | Example | Example | Example |
|---|---|---|---|---|---|---|---|---|---|
| | | 1 | 2 | 3 | 4 | 5 | 6 | 7 | 8 |
| (A) | Hydrogenated | 2.00 | 2.00 | 2.00 | 1.00 | 2.00 | 2.50 | 3.00 | 1.00 |
| | castor oil | | | | | | | | |
| (B) | Polyethylene wax A | 4.00 | 4.00 | 4.00 | 5.00 | 5.00 | 5.00 | 3.00 | 3.00 |
| | Polyethylene wax B | — | — | — | — | — | — | — | — |
| (C) | Ethylhexyl | 64.00 | 64.00 | 64.00 | 64.00 | 63.00 | 62.50 | 64.00 | 64.00 |
| | palmitate | | | | | | | | |
| | Triethylhexanoin | 20.00 | — | — | 20.00 | 20.00 | 20.00 | 20.00 | 20.00 |
| | Glyceryl | — | 20.00 | — | — | — | — | — | — |
| | tri(caprylate/caprate) | | | | | | | | |
| | Isostearyl isostearate | — | — | 20.00 | — | — | — | — | — |
| (D) | PEG-20 glyceryl | 10.00 | 10.00 | 10.00 | 10.00 | 10.00 | 10.00 | 10.00 | 10.00 |
| | triisostearate | | | | | | | | |
| Evaluation | Initial hardness (N) | 0.67 | 0.88 | 0.83 | 0.85 | 1.08 | 1.29 | 1.01 | 0.38 |
| | Usability | A | A | A | A | B | C | B | B |
| | (ease of taking out) | | | | | | | | |
| | Usability | A | A | A | A | A | B | B | A |
| | (makeup compatibility) | | | | | | | | |
| | Usability | A | A | B | A | A | B | B | A |
| | (absence of sliminess) | | | | | | | | |
| | Storage stability | A | A | A | A | A | A | A | A |

TABLE 2

| | | Example 9 | Example 10 | Example 11 | Example 12 | Example 13 | Example 14 | Example 15 |
|---|---|---|---|---|---|---|---|---|
| (A) | Hydrogenated | 2.00 | 2.00 | 5.00 | 4.00 | 2.00 | 2.00 | 2.00 |
| | castor oil | | | | | | | |
| (B) | Polyethylene wax A | — | 2.00 | 1.00 | 2.00 | 4.00 | 4.00 | 4.00 |
| | Polyethylene wax B | 4.00 | 2.00 | — | — | — | — | — |
| (C) | Ethylhexyl | 64.00 | 64.00 | 64.00 | 64.00 | 20.00 | 64.00 | 64.00 |
| | palmitate | | | | | | | |
| | Triethyl hexanoin | 20.00 | 20.00 | 20.00 | 20.00 | 64.00 | — | — |
| Liquid oil | Dimethicone | — | — | — | — | — | 20.00 | |
| other than | Diphenylsiloxyphenyltrimethicone | — | — | — | — | — | — | 20.00 |
| (C) | | | | | | | | |
| (D) | PEG-20 glyceryl | 10.00 | 10.00 | 10.00 | 10.00 | 10.00 | 10.00 | 10.00 |
| | triisostearate | | | | | | | |
| Evaluation | Initial hardness (N) | 0.74 | 0.63 | 1.42 | 1.07 | 0.95 | 0.88 | 0.83 |
| | Usability | A | A | C | B | A | A | A |
| | (ease of taking out) | | | | | | | |
| | Usability | A | A | B | B | B | A | A |
| | (makeup compatibility) | | | | | | | |
| | Usability | A | A | B | B | C | A | A |
| | (absence of sliminess) | | | | | | | |
| | Storage stability | A | A | A | A | A | A | A |

TABLE 3

| | | Comparative | Comparative | Comparative | Comparative | Comparative | Comparative | Comparative |
|---|---|---|---|---|---|---|---|---|
| | | Example 1 | Example 2 | Example 3 | Example 4 | Example 5 | Example 6 | Example 7 |
| (A) | Hydrogenated | 3.00 | — | — | — | — | — | — |
| | castor oil | | | | | | | |
| (B) | Polyethylene wax A | 5.00 | 6.00 | 4.00 | 4.00 | 2.00 | 1.00 | 4.00 |
| | Candelilla wax | — | — | 2.00 | — | — | — | — |
| | Carnauba wax | — | — | — | 2.00 | — | — | — |
| | Dextrin palmitate | — | — | — | — | 4.00 | 5.00 | 2.00 |
| (C) | Ethylhexyl | 62.00 | 64.00 | 64.00 | 64.00 | 64.00 | 64.00 | 64.00 |
| | palmitate | | | | | | | |
| | Triethylhexanoin | 20.00 | 20.00 | 20.00 | 20.00 | 20.00 | 20.00 | 20.00 |
| (D) | PEG-20 glyceryl | 10.00 | 10.00 | 10.00 | 10.00 | 10.00 | 10.00 | 10.00 |
| | triisostearate | | | | | | | |
| Evaluation | Initial hardness (N) | 1.72 | 1.07 | 0.38 | 1.05 | 0.03 | 0.05 | 0.07 |
| | Usability | D | C | B | C | D | D | D |
| | (ease of taking out) | | | | | | | |
| | Usability | D | C | B | B | A | A | A |
| | (makeup compatibility) | | | | | | | |
| | Usability | B | B | B | B | D | D | D |
| | (absence of sliminess) | | | | | | | |
| | Storage stability | A | A | C | B | A | A | C |

### Example 16

| | (Mixing proportion |
|---|---|
| (Component) | (mass %)) |
| 1. Hydrogenated castor oil | 1.5 |
| 2. Polyethylene wax A | 4.0 |
| 3. Ethylhexyl palmitate | 61.5 |
| 4. Octyldodecanol | 20.0 |
| 5. PEG-20 glyceryl triisostearate | 13.0 |

The details of the respective components are as described above. Incidentally, the viscosity at 25° C. of octyldodecanol is 49 mPa·s.

<Production Method>

The components of 1 to 5 were mixed at 105° C. using a Disper, and an oily solid cleansing cosmetic was obtained.

<Evaluation>

For the oily solid cleansing cosmetic thus obtained, the above-described evaluations were carried out, and the results were as follows: initial hardness 0.66 N, usability (ease of taking out “A”, makeup compatibility “B”, absence of sliminess “A”), and storage stability “A”.

What is claimed is:

1. An oily solid cleansing cosmetic containing: (A) hydrogenated castor oil, (B) a hydrocarbon-based wax, (C) an ester oil whose viscosity at 25° C. is 50 mPa·s or less, and (D) a nonionic surfactant,
wherein the content of the component (A) is 1% to 5% by mass,
the content of the component (B) is 1% to 5% by mass,
the content of the component (C) is 60% by mass or more, and
the total content of the component (A) and the component (B) is 4% to 7.5% by mass.

2. The oily solid cleansing cosmetic according to claim 1, wherein the total content of the component (A), the component (B), the component (C), and the component (D) is 97% by mass or more.

3. The oily solid cleansing cosmetic according to claim 1, wherein 50% by mass or more of the component (C) is an ester oil whose viscosity at 25° C. is 20 mPa·s or less.

4. The oily solid cleansing cosmetic according to claim 1, wherein the component (B) is a polyethylene wax having a melting point of 70° C. to 110° C.

5. The oily solid cleansing cosmetic according to claim 1, wherein the mass ratio (A)/(B) of the component (A) and the component (B) is 1/5 to 1/1.

6. The oily solid cleansing cosmetic according to claim 1, wherein the hardness at 25° C. is 0.3 to 1.2 N.

7. The oily solid cleansing cosmetic according to claim 1, wherein the component (C) contains a first ester oil whose viscosity at 25° C. is 1 to 15 mPa·s and a second ester oil whose viscosity at 25° C. is 16 to 50 mPa·s, and
the mass ratio between the first ester oil and the second ester oil, (first ester oil)/(second ester oil), is 1/1 to 4/1.

8. The oily solid cleansing cosmetic according to claim 1, wherein the oily solid cleansing cosmetic contains one kind or two or more kinds of liquid oil other than the component (C) selected from the group consisting of an aliphatic alcohol and a silicone oil, and
the mass ratio between the component (C) and the liquid oil other than the component (C), (component (C))/(liquid oil other than component (C)), is 2/1 to 20/1.

9. The oily solid cleansing cosmetic according to claim 1, wherein
the total content of the component (A), the component (B), the component (C), and the component (D) is 97% by mass or more,
the mass ratio (A)/(B) of the component (A) and the component (B) is 1/5 to 1/1, and
50% by mass or more of the component (C) is an ester oil whose viscosity at 25° C. is 20 mPa·s or less.
